(19) United States
(12) Patent Application Publication
(10) Pub. No.: US 2015/0099953 A1
(43) Pub. Date: Apr. 9, 2015

(54) METHODS AND SYSTEMS FOR TRIGGERING PHYSIOLOGICAL MEASUREMENTS

(71) Applicant: Covidien LP, Mansfield, MA (US)

(72) Inventor: Clark R. Baker, JR., Newman, CA (US)

(21) Appl. No.: 14/509,823

(22) Filed: Oct. 8, 2014

Related U.S. Application Data
(60) Provisional application No. 61/888,431, filed on Oct. 8, 2013.

(51) Int. Cl. A61B 5/1455; A61B 5/021

(52) U.S. Cl. CPC A61B 5/14552; A61B 5/02141
USPC 600/324

(57) ABSTRACT

Methods and systems are presented for triggering physiological measurements in a physiological monitor. Metrics are computed for a received physiological signal (e.g., a PPG signal), or a determined physiological parameter associated with the physiological signal (e.g., blood pressure). A change parameter is determined based on one or more of the metrics, and a variable change threshold is determined. The variable change threshold may be determined over time based on a time measure, a frequency measure, or both. The change parameter is compared to the variable change threshold, and a physiological measurement is triggered based on the comparison. The variable change threshold technique may allow measurements to be taken frequently enough to catch clinically significant changes in a physiological parameter of a subject but not so often as to interfere with the subject's comfort or the function of other medical monitors.

### CROSS REFERENCE TO RELATED APPLICATION

This application claims the benefit of U.S. Provisional Application No. 61/888,431, filed Oct. 8, 2013, which is hereby incorporated by reference herein in its entirety.

### SUMMARY

The present disclosure relates to triggering physiological measurements in a physiological monitor, and more particularly, relates to triggering blood pressure measurements in an oximeter or other medical device.

Methods and systems are provided for triggering physiological measurements. In some embodiments, the physiological monitor of the present disclosure may be a non-invasive blood pressure monitoring system. Conventional non-invasive blood pressure (NIBP) monitoring systems trigger cuff inflation for direct measurement of blood pressure at fixed intervals, which may be specified by the clinician. If the interval used is too long, clinically important changes in the subject's blood pressure may be missed. If the interval is too short, repeated cuff inflations may disrupt the signal to other medical monitors, for example, a pulse oximeter, or cause the subject excessive discomfort. Accordingly, the physiological monitor of the present disclosure may trigger physiological measurements based on a dynamically determined change threshold, which allows for measurements to be taken at more appropriate times. For example, cuff inflations may be triggered when a subject exhibits sufficiently significant changes associated with a PPG signal or a determined blood pressure measurement. Thus, the cuff inflations may be triggered frequently enough to catch clinically significant changes but not so often as to interfere with the subject's comfort or the function of other medical monitors.

In some embodiments, a system receives a physiological signal from a subject, for example, a photoplethysmograph (PPG) signal, and processes it to determine a change parameter of the physiological signal over time. The system determines a variable change threshold for the received physiological signal over time. The system uses a comparison of the change parameter and the variable change threshold to trigger a physiological measurement.

In some embodiments, a method for triggering a physiological measurement of a subject includes receiving a physiological signal from the subject and determining a change parameter based at least in part on the received physiological signal over time. The method includes determining a variable change threshold over time. The method includes comparing the change parameter with the variable change threshold and triggering a measurement of a physiological parameter based on the comparison.

In some embodiments, a system for triggering a physiological measurement of a subject includes an input configured for receiving a physiological signal from the subject and one or more processors configured for determining a change parameter based at least in part on the received physiological signal over time. The one or more processors are further configured for determining a variable change threshold over time. The one or more processors are further configured for comparing the change parameter with the variable change threshold and triggering a measurement of a physiological parameter based on the comparison.

### BRIEF DESCRIPTION OF THE FIGURES

The above and other features of the present disclosure, its nature and various advantages will be more apparent upon consideration of the following detailed description, taken in conjunction with the accompanying drawings in which:

FIG. 1 is a block diagram of an illustrative physiological monitoring system in accordance with some embodiments of the present disclosure;

FIG. 2 is a perspective view of an illustrative physiological monitoring system in accordance with some embodiments of the present disclosure;

FIG. 3 shows an illustrative plot of a signal that may be analyzed in accordance with some embodiments of the present disclosure;

FIGS. 4A and 4B show illustrative flow diagrams including steps for determining a change parameter in accordance with some embodiments of the present disclosure;

FIG. 5 shows an illustrative flow diagram including steps for determining a variable change threshold over time in accordance with some embodiments of the present disclosure;

FIG. 6 is an illustrative block diagram for updating a variable change threshold in accordance with some embodiments of the present disclosure.

FIG. 7 shows an illustrative plot of a variable change threshold in accordance with some embodiments of the present disclosure;

FIG. 8 shows an illustrative plot of a variable change threshold in accordance with some embodiments of the present disclosure; and

FIG. 9 shows an illustrative flow diagram including steps for triggering a physiological measurement in accordance with some embodiments of the present disclosure.

### DETAILED DESCRIPTION OF THE FIGURES

The present disclosure is directed towards triggering physiological measurements in a physiological monitor. The physiological monitor may determine one or more metrics for a received physiological signal (e.g., a PPG signal). For example, a metric corresponding to a change in blood pressure may be determined. The physiological monitor may determine a change parameter based on one or more of the metrics and a variable change threshold over time. The variable change threshold may be determined based on a time measure, indicative of time passed since a physiological measurement was triggered, and/or a frequency measure, indicative of how frequently the measurement was triggered. The change parameter may be compared to the current variable change threshold, and a physiological measurement may be triggered based on the comparison.

In some embodiments, the physiological monitor of the present disclosure may be a non-invasive blood pressure monitoring system such as a continuous non-invasive blood pressure (CNIBP) monitoring system. CNIBP monitoring systems continuously measure a subject's blood pressure but typically require periodic recalibration. The calibration may occur periodically at fixed intervals or as specified by a clinician. The physiological monitor of the present disclosure may recalibrate the continuous blood pressure measurement based on a triggered NIBP measurement. In some embodiments, pulse oximeters may be utilized in a CNIBP monitoring system, as described in detail below.

In some embodiments, the physiological monitor of the present disclosure may be an oximeter. An oximeter is a medical device that may determine the oxygen saturation of the blood. One common type of oximeter is a pulse oximeter, which may indirectly measure the oxygen saturation of a subject's blood (as opposed to measuring oxygen saturation directly by analyzing a blood sample taken from the subject). Pulse oximeters may be included in physiological monitoring systems that measure and display various blood flow characteristics including, but not limited to, the oxygen saturation of hemoglobin in arterial blood. Such physiological monitoring systems may also measure and display additional physiological parameters, such as a subject's pulse rate, respiration rate, and blood pressure.

An oximeter may include a light sensor that is placed at a site on a subject, typically a fingertip, toe, forehead or earlobe, or in the case of a neonate, across a foot. The oximeter may use a light source to pass light through blood perfused tissue and photoelectrically sense the transmission of the light in the tissue. In addition, locations which are not typically understood to be optimal for pulse oximetry may serve as suitable sensor locations for the blood pressure monitoring processes described herein, including any location on the body that has a strong pulsatile arterial flow. For example, additional suitable sensor locations include, without limitation, the neck to monitor carotid artery pulsatile flow, the wrist to monitor radial artery pulsatile flow, the inside of a subject's thigh to monitor femoral artery pulsatile flow, the ankle to monitor tibial artery pulsatile flow, and around or in front of the ear. Suitable sensors for these locations may include sensors for sensing attenuated light based on detecting reflected light. In all suitable locations, for example, the oximeter may measure the intensity of light that is received at the light sensor as a function of time. The oximeter may also include sensors at multiple locations. A signal representing light intensity versus time or a mathematical manipulation of this signal (e.g., a scaled version thereof, a log taken thereof, a scaled version of a log taken thereof, etc.) may be referred to as a photoplethysmograph (PPG) signal. In addition, the term “PPG signal,” as used herein, may also refer to an absorption signal (i.e., representing the amount of light absorbed by the tissue) or any suitable mathematical manipulation thereof. The light intensity or the amount of light absorbed may then be used to calculate any of a number of physiological parameters, including an amount of a blood constituent (e.g., oxyhemoglobin) being measured as well as a pulse rate and when each individual pulse occurs.

In some applications, the light passed through the tissue is selected to be of one or more wavelengths that are absorbed by the blood in an amount representative of the amount of the blood constituent present in the blood. The amount of light passed through the tissue varies in accordance with the changing amount of blood constituent in the tissue and the related light absorption. Red and infrared (IR) wavelengths may be used because it has been observed that highly oxygenated blood will absorb relatively less Red light and more IR light than blood with a lower oxygen saturation. By comparing the intensities of two wavelengths at different points in the pulse cycle, it is possible to estimate the blood oxygen saturation of hemoglobin in arterial blood.

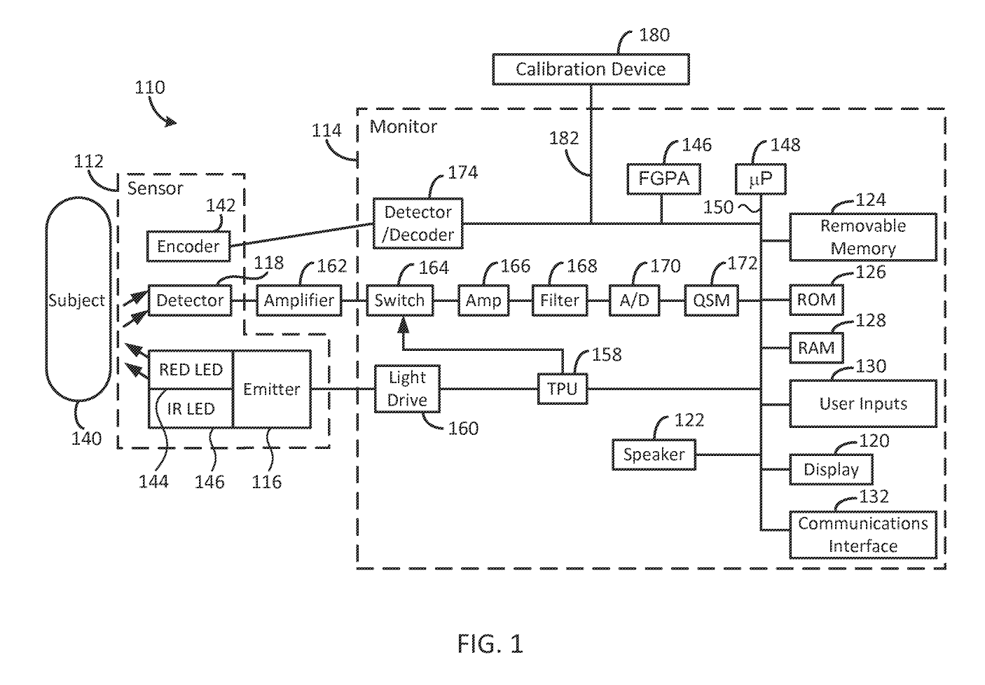

FIG. 1 is a block diagram of an illustrative physiological monitoring system 110 in accordance with some embodiments of the present disclosure. System 110 may include a sensor 112 and a monitor 114 for generating and processing physiological signals of a subject 140. In some embodiments, system 110 may be coupled to subject 140. In some embodiments, sensor 112 and monitor 114 may be part of a blood pressure monitoring system and/or an oximeter.

Sensor unit 112 may include emitter 116, detector 118, and encoder 142. In the embodiment shown, emitter 116 may be configured to emit at least two wavelengths of light (e.g., red and IR) into the tissue of subject 140. For example, in the embodiment shown, emitter 116 may include a red light emitting light source such as RED light emitting diode (LED) 144 and an IR light emitting light source such as IR LED 146 for emitting light into the tissue of subject 140 to generate physiological signals. In some embodiments, the red wavelength may be between about 600 nm and about 700 nm, and the IR wavelength may be between about 800 nm and about 1000 nm. It will be understood that emitter 116 may include any number of light sources with any suitable characteristics. In embodiments where an array of sensors is used in place of single sensor 112, each sensor may be configured to emit a single wavelength. For example, a first sensor may emit only a red light while a second may emit only an IR light. In another example, the wavelengths of light used are selected based on the specific location of the sensor.

It will be understood that, as used herein, the term “light” may refer to energy produced by radiative sources and may include one or more of ultrasound, radio, microwave, millimeter wave, infrared, visible, ultraviolet, gamma ray or X-ray electromagnetic radiation. As used herein, light may also include any wavelength within the radio, microwave, infrared, visible, ultraviolet, or X-ray spectra, and that any suitable wavelength of electromagnetic radiation may be appropriate for use with the present techniques. Detector 118 may be chosen to be specifically sensitive to the chosen targeted energy spectrum of the emitter 116, the hemoglobin absorption profile, or both.

In some embodiments, detector 118 may be configured to detect the intensity of light at the red and IR wavelengths. In some embodiments, an array of sensors may be used and each sensor in the array may be configured to detect an intensity of a single wavelength. In operation, light may enter detector 118 after being attenuated (e.g., absorbed, scattered) by the tissue of subject 140. Detector 118 may convert the intensity of the received light into an electrical signal. The light intensity may be directly related to the absorbance and/or reflectance of light in the tissue. That is, when more light at a certain wavelength is absorbed or reflected, less light of that wavelength is received from the tissue by detector 118. After converting the received light to an electrical signal, detector 118 may send the signal to monitor 114, where the signal may be processed and physiological parameters may be determined (e.g., based on the absorption of the red and IR wavelengths in the tissue of subject 140).

In some embodiments, encoder 142 may contain information about sensor 112, such as sensor type (e.g., whether the sensor is intended for placement on a forehead or digit), the wavelengths of light emitted by emitter 116, power requirements or limitations of emitter 116, or other suitable information. This information may be used by monitor 114 to select appropriate algorithms, lookup tables and/or calibration coefficients stored in monitor 114 for calculating the subject's physiological parameters.

In some embodiments, encoder 142 may contain information specific to subject 140, such as, for example, the subject's age, weight, and diagnosis. Information regarding a subject's characteristics may allow monitor 114 to determine, for example, subject-specific threshold ranges in which the subject's physiological parameter measurements should fall and to enable or disable additional physiological parameter algorithms. This information may also be used to select and provide coefficients for equations from which, for example, oxygen saturation, pulse rate, blood pressure, and other measurements may be determined based on the signal or signals received at sensor unit 112. For example, some pulse oximetry sensors rely on equations to relate an area under a portion of a PPG signal corresponding to a physiological pulse to determine blood pressure. These equations may contain coefficients that depend upon a subject's physiological characteristics as stored in encoder 142. Encoder 142 may, for instance, be a coded resistor which stores values corresponding to the type of sensor unit 112 or the type of each sensor in the sensor array, the wavelengths of light emitted by emitter 116 on each sensor of the sensor array, and/or the subject's characteristics. In some embodiments, encoder 142 may include a memory on which one or more of the following information may be stored for communication to monitor 114: the type of the sensor unit 112; the wavelengths of light emitted by emitter 116; the particular wavelength each sensor in the sensor array is monitoring; a signal threshold for each sensor in the sensor array; any other suitable information; or any combination thereof. In some embodiments, encoder 142 may include an identifying component such as, for example, a radio-frequency identification (RFID) tag that may be read by decoder 174.

In some embodiments, signals from detector 118 and encoder 142 may be transmitted to monitor 114. In the embodiment shown, monitor 114 may include a general purpose microprocessor 148, FPGA 146, or both, connected to an internal bus 150. In some embodiments, monitor 114 may include one or more microprocessors, digital signal processors (DSPs), or both. Microprocessor 148 may be adapted to execute software, which may include an operating system and one or more applications, as part of performing the functions described herein. Also connected to bus 150 may be a read-only memory (ROM) 126, a random access memory (RAM) 128, removable memory 124, user inputs 130, display 120, and speaker 122.

RAM 128, ROM 126, and removable memory 124 are provided as illustrative examples (e.g., communications interface 132, flash memory, digital logic array, field programmable gate array (FPGA), or any other suitable memory) and are not provided by way of limitation. Any suitable computer-readable media may be used in the system for data storage. Computer-readable media are capable of storing information that can be interpreted by microprocessor 148, FPGA 146, or both. This information may be data or may take the form of computer-executable instructions, such as software applications, that cause the microprocessor to perform certain functions and/or computer-implemented methods. Depending on the embodiment, such computer-readable media may include computer storage media and communication media. Computer storage media may include volatile and non-volatile, writable and non-writable, and removable and non-removable media implemented in any method or technology for storage of information such as computer-readable instructions, data structures, program modules or other data. Computer storage media may include, but is not limited to, RAM, ROM, EPROM, EEPROM, flash memory or other solid state memory technology, CD-ROM, DVD, or other optical storage, magnetic cassettes, magnetic tape, magnetic disk storage or other magnetic storage devices, or any other medium which can be used to store the desired information and which can be accessed by components of the system.

In the embodiment shown, a time processing unit (TPU) 158 may provide timing control signals to light drive circuitry 160, which may control when emitter 116 is illuminated and multiplexed timing for RED LED 144 and IR LED 146. TPU 158 may also control the gating-in of signals from detector 118 through amplifier 162 and switching circuit 164. These signals are sampled at the proper time, depending upon which light source is illuminated. In some embodiments, microprocessor 148, FPGA 146, or both, may de-multiplex the signal from detector 118 using de-multiplexing techniques such as time-division, frequency-division, code division, or any other suitable de-multiplexing technique. In some embodiments, microprocessor 148, FPGA 146, or both, may perform the functions of TPU 158 using suitable timing signals and multiplexing/de-multiplexing algorithms, and accordingly TPU 158 need not be included. The received signal from detector 118 may be passed through amplifier 166, low pass filter 168, and analog-to-digital converter 170. The digital data may then be stored in a queued serial module (QSM) 172 (or buffer such as a first in first out (FIFO) buffer) for later downloading to RAM 128 as QSM 172 fills up. A window of data may be selected from the data stored in the buffer for further processing. In some embodiments, there may be multiple separate parallel paths having components equivalent to amplifier 162, switching circuit 164, amplifier 166, filter 168, and/or A/D converter 170 for multiple light wavelengths or spectra received. In some embodiments, a filter (e.g., an analog filter) may be included (not shown) between amplifier 162 and switching circuit 164.

In some embodiments, microprocessor 148 may determine the subject's physiological parameters, such as pulse rate, SpO₂, and/or blood pressure, using various algorithms and/or look-up tables based on the value of the received signals and/or data corresponding to the light received by detector 118. Signals corresponding to information about subject 140, and particularly about the intensity of attenuated light emanating from a subject's tissue over time, may be transmitted from encoder 142 to decoder 174. These signals may include, for example, encoded information relating to subject characteristics. Decoder 174 may translate these signals to enable the microprocessor to determine the thresholds based on algorithms or look-up tables stored in ROM 126. In some embodiments, user inputs 130 may be used to enter information, select one or more options, provide a response, input settings, any other suitable inputting function, or any combination thereof. User inputs 130 may be used to enter information about the subject, such as age, weight, height, diagnosis, medications, treatments, and so forth. In some embodiments, display 120 may exhibit a list of values which may generally apply to the subject, such as, for example, age ranges or medication families, which the user may select using user inputs 130.

Calibration device 180, which may be powered by monitor 114 via a coupling 182, a battery, or by a conventional power source such as a wall outlet, may include any suitable signal calibration device. Calibration device 180 may be communicatively coupled to monitor 114 via communicative coupling 182, and/or may communicate wirelessly (not shown). In some embodiments, calibration device 180 is completely integrated within monitor 114. In some embodiments, calibration device 180 may include a manual input device (not shown) used by an operator to manually input reference signal measurements obtained from some other source (e.g., an external invasive or non-invasive physiological measurement system). Calibration device 180 may be coupled to one or more components of monitor 114 to calibrate monitor 114.

Communications interface 132 may enable monitor 114 to exchange information with external devices. Communications interface 132 may include any suitable hardware, software, or both, which may allow physiological monitoring system 110 (e.g., monitor 114) to communicate with electronic circuitry, a device, a network, or any combinations thereof. Communications interface 132 may include one or more receivers, transmitters, transceivers, antennas, plug-in connectors, ports, communications buses, communications protocols, device identification protocols, any other suitable hardware or software, or any combination thereof. Communications interface 132 may be configured to allow wired communication (e.g., using USB, RS-232, Ethernet, or other standards), wireless communication (e.g., using WiFi, IR, WiMax, BLUETOOTH, UWB, or other standards), or both. For example, communications interface 132 may be configured using a universal serial bus (USB) protocol (e.g., USB 2.0, USB 3.0), and may be configured to couple to other devices (e.g., remote memory devices storing templates) using a four-pin USB standard Type-A connector (e.g., plug and/or socket) and cable. In some embodiments, communications interface 132 may include an internal bus such as, for example, one or more slots for insertion of expansion cards.

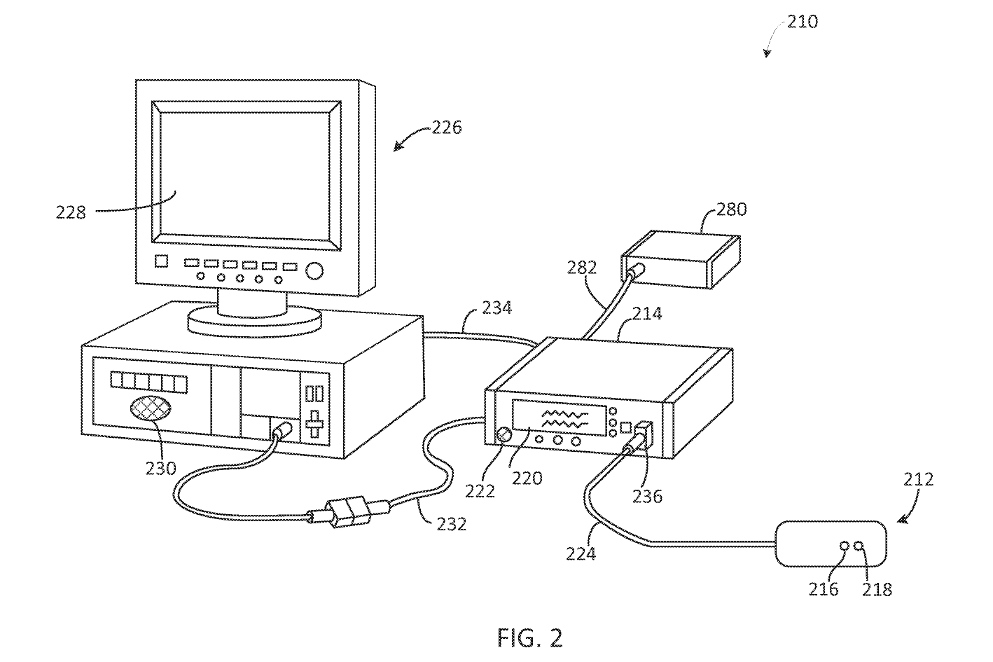

FIG. 2 is a perspective view of an illustrative physiological monitoring system 210 in accordance with some embodiments of the present disclosure. In some embodiments, one or more components of physiological monitoring system 210 may include one or more components of physiological monitoring system 110 of FIG. 1. Physiological monitoring system 210 may include sensor unit 212 and monitor 214. In some embodiments, sensor unit 212 may be part of a continuous, non-invasive blood pressure (CNIBP) monitoring system and/or an oximeter. Sensor unit 212 may include light source 216 for emitting light at one or more wavelengths into a subject's tissue. Detector 218 may also be provided in sensor unit 212 for detecting the light that is reflected by or has traveled through the subject's tissue. Any suitable configuration of light source 216 and detector 218 may be used. In some embodiments, sensor unit 212 may include multiple light sources and detectors, which may be spaced apart. Physiological monitoring system 210 may also include one or more additional sensor units (not shown) that may, for example, take the form of any of the embodiments described herein with reference to sensor unit 212. An additional sensor unit may be the same type of sensor unit as sensor unit 212, or a different sensor unit type than sensor unit 212. Multiple sensor units may be capable of being positioned at two different locations on a subject's body. For example, a first sensor unit may be positioned on a subject's forehead, while a second sensor unit may be positioned at a subject's fingertip.

Sensor units may each detect any signal that carries information about a subject's physiological state, such as an electrocardiograph signal, arterial line measurements, or the pulsatile force exerted on the walls of an artery using, for example, oscillometric methods with a piezoelectric transducer. According to another embodiment, physiological monitoring system 210 may include a plurality of sensors forming a sensor array in lieu of either or both of the sensor units. Each of the sensors of a sensor array may be a complementary metal oxide semiconductor (CMOS) sensor. Alternatively, each sensor of an array may be a charged coupled device (CCD) sensor. In some embodiments, a sensor array may be made up of a combination of CMOS and CCD sensors. The CCD sensor may comprise a photoactive region and a transmission region for receiving and transmitting data whereas the CMOS sensor may be made up of an integrated circuit having an array of pixel sensors. In some embodiments, each pixel may have a photodetector and an active amplifier. In some embodiments, a group of pixels may share an amplifier. It will be understood that any type of sensor, including any type of physiological sensor, may be used in one or more sensor units in accordance with the systems and techniques disclosed herein. It is understood that any number of sensors measuring any number of physiological signals may be used to determine physiological information in accordance with the techniques described herein.

In some embodiments, light source 216 and detector 218 may be on opposite sides of a digit such as a finger or toe, in which case the light that is emanating from the tissue has passed completely through the digit. In some embodiments, light source 216 and detector 218 may be arranged so that light from light source 216 penetrates the tissue and is attenuated by the tissue and transmitted to detector 218, such as in a sensor designed to obtain pulse oximetry data from a subject's forehead.

In some embodiments, sensor unit 212 may be connected to and draw its power from monitor 214 as shown. In some embodiments, sensor unit 212 may be wirelessly connected (not shown) to monitor 214 and may be powered by an internal power source such as a battery (not shown). Monitor 214 may be configured to calculate physiological parameters based at least in part on data relating to light emission and detection received from one or more sensor units such as sensor unit 212. For example, monitor 214 may be configured to determine pulse rate, respiration rate, respiration effort, blood pressure, blood oxygen saturation (e.g., arterial, venous, or both), hemoglobin concentration (e.g., oxygenated, deoxygenated, and/or total), any other suitable physiological parameters, or any combination thereof. In some embodiments, calculations may be performed on the sensor units or an intermediate device and the result of the calculations may be passed to monitor 214. Further, monitor 214 may include display 220 configured to display the physiological parameters or other information about the system. In the embodiment shown, monitor 214 may also include a speaker 222 to provide an audible sound that may be used in various other embodiments, such as for example, sounding an audible alarm in the event that a subject's physiological parameters are not within a predefined normal range. In some embodiments, monitor 214 may include a blood pressure monitor. In some embodiments, the physiological monitoring system 210 may include a stand-alone blood pressure monitor in communication with the monitor 214 via a cable or a wireless network link. In some embodiments, monitor 214 may be implemented as display 120 of FIG. 1.

In some embodiments, sensor unit 212 may be communicatively coupled to monitor 214 via a cable 224 at port 236. Cable 224 may include electronic conductors (e.g., wires for transmitting electronic signals from detector 218), optical fibers (e.g., multi-mode or single-mode fibers for transmitting emitted light from light source 216), any other suitable components, any suitable insulation or sheathing, or any combination thereof. In some embodiments, a wireless transmission device (not shown) or the like may be used instead of or in addition to cable 224. Monitor 214 may include a sensor interface configured to receive physiological signals from sensor unit 212, provide signals and power to sensor unit 212, or otherwise communicate with sensor unit 212. The sensor interface may include any suitable hardware, software, or both, which may allow communication between monitor 214 and sensor unit 212.

In the illustrated embodiment, physiological monitoring system 210 includes a multi-parameter physiological monitor 226. Multi-parameter physiological monitor 226 may include a cathode ray tube display, a flat panel display (as shown) such as a liquid crystal display (LCD) or a plasma display, or may include any other type of monitor now known or later developed. Multi-parameter physiological monitor 226 may be configured to calculate physiological parameters and to provide a display 228 for information from monitor 214 and from other medical monitoring devices or systems (not shown). For example, multi-parameter physiological monitor 226 may be configured to display pulse rate information from monitor 214, an estimate of a subject's blood oxygen saturation generated by monitor 214, and blood pressure from monitor 214 on display 228. Multi-parameter physiological monitor 226 may include a speaker 230.

Monitor 214 may be communicatively coupled to multi-parameter physiological monitor 226 via a cable 232 or 234 that is coupled to a sensor input port or a digital communications port, respectively and/or may communicate wirelessly (not shown). In addition, monitor 214 and/or multi-parameter physiological monitor 226 may be coupled to a network to enable the sharing of information with servers or other workstations (not shown). Monitor 214 may be powered by a battery (not shown) or by a conventional power source such as a wall outlet.

In some embodiments, physiological monitoring system 210 may include calibration device 280. Calibration device 280, which may be powered by monitor 214, a battery, or by a conventional power source such as a wall outlet, may include any suitable calibration device. Calibration device 280 may be communicatively coupled to monitor 214 via communicative coupling 282, and/or may communicate wirelessly (not shown). In some embodiments, calibration device 280 may be completely integrated within monitor 214. For example, calibration device 280 may take the form of any invasive or non-invasive blood pressure monitoring or measuring system used to generate reference blood pressure measurements for use in calibrating a CNIBP monitoring technique as described herein. Such calibration devices may include, for example, an aneroid or mercury sphygmomanometer and occluding cuff, a pressure sensor inserted directly into a suitable artery of a subject, an oscillometric device or any other device or mechanism used to sense, measure, determine, or derive a reference blood pressure measurement. In some embodiments, calibration device 280 may include a manual input device (not shown) used by an operator to manually input reference signal measurements obtained from some other source (e.g., an external invasive or non-invasive physiological measurement system).

Calibration device 280 may also access reference signal measurements stored in memory (e.g., RAM, ROM, or a storage device). For example, in some embodiments, calibration device 280 may access reference blood pressure measurements from a relational database stored within calibration device 280, monitor 214, or multi-parameter physiological monitor 226. The reference blood pressure measurements generated or accessed by calibration device 280 may be updated in real-time, resulting in a continuous source of reference blood pressure measurements for use in continuous or periodic calibration. Alternatively, reference blood pressure measurements generated or accessed by calibration device 280 may be updated periodically, and calibration may be performed on the same periodic cycle or a different periodic cycle. In some embodiments, reference blood pressure measurements may be generated when recalibration is triggered. For example, recalibration may be triggered based on a change parameter.

In some embodiments, any of the processing components and/or circuits, or portions thereof, of FIGS. 1 and 2, including sensors 112 and 212 and monitors 114, 214, and 226 may be referred to collectively as processing equipment. For example, processing equipment may be configured to amplify, filter, sample and digitize an input signal from sensor 112 or 212 (e.g., using an analog-to-digital converter), calculate metrics from the digitized signal, and trigger a physiological measurement or recalibration. In some embodiments, all or some of the components of the processing equipment may be referred to as a processing module.

The optical signal attenuated by the tissue can be degraded by noise, among other sources, and an electrical signal derived thereof can also be degraded by noise. One source of noise is ambient light that reaches the light detector. Another source of noise in an intensity signal is electromagnetic coupling from other electronic instruments. Movement of the subject also introduces noise and affects the signal. For example, the contact between the detector and the skin, or the emitter and the skin, can be temporarily disrupted when movement causes either to move away from the skin. In addition, because blood is a fluid, it responds differently than the surrounding tissue to inertial and pressure effects, thus resulting in momentary changes in volume at the point to which the oximeter probe is attached.

Noise (e.g., from subject movement) can degrade a sensor signal relied upon by a care provider, without the care provider's awareness. This is especially true if the monitoring of the subject is remote, the motion is too small to be observed, or the care provider is watching the instrument or other parts of the subject, and not the sensor site. Analog and/or digital processing of sensor signals (e.g., PPG signals) may involve operations that reduce the amount of noise present in the signals or otherwise identify noise components in order to prevent them from affecting measurements of physiological parameters derived from the sensor signals.

It will be understood that the present disclosure is applicable to any suitable signal and that PPG signals are used merely for illustrative purposes. Those skilled in the art will recognize that the present disclosure has wide applicability to other signals including, but not limited to, other biosignals (e.g., electrocardiograms, electroencephalograms, electrogastrograms, electromyograms, pulse rate signals, pathological signals, ultrasound signals, any other suitable biosignals), or any combination thereof.

Pulse oximeters can be utilized for continuous non-invasive blood pressure monitoring. As described in Chen et al., U.S. Pat. No. 6,599,251, the entirety of which is incorporated herein by reference, PPG and other pulse signals obtained from multiple probes can be processed to calculate the blood pressure of a subject. In particular, blood pressure measurements may be derived based on a comparison of time differences between certain components of the pulse signals detected at each of the respective probes. As described in U.S. Patent Publication No. 2009/0326386, published Dec. 31, 2009, the entirety of which is incorporated herein by reference, blood pressure can also be derived by processing time delays detected within a single PPG or pulse signal obtained from a single pulse oximeter probe. In addition, as described in U.S. Pat. No. 8,398,556, the entirety of which is incorporated herein by reference, blood pressure may also be obtained by calculating the area under certain portions of a pulse signal. Finally, as described in U.S. Patent Application Publication No. 2010/0081945, published Apr. 1, 2010, the entirety of which is incorporated herein by reference, a blood pressure monitoring device may be recalibrated in response to arterial compliance changes.

As described above, some CNIBP monitoring techniques utilize two probes or sensors positioned at two different locations on a subject's body. The elapsed time, T, between the arrivals of corresponding points of a pulse signal at the two locations may then be determined using signals obtained by the two probes or sensors. The estimated blood pressure, p, may then be related to the elapsed time, T, by:

p=a+b·ln(T), (1)

where a and b are constants that may be dependent upon the nature of the subject and the nature of the signal detecting devices. Other suitable equations using an elapsed time between corresponding points of a pulse signal may also be used to derive an estimated blood pressure measurement.

In some embodiments, Eq. 1 may include a nonlinear function which is monotonically decreasing and concave upward in T in a manner specified by the constant parameters (in addition to or instead of the expression of Eq. 1). Eq. 1 may be used to calculate an estimated blood pressure from the time difference T between corresponding points of a pulse signal received by two sensors or probes attached to two different locations of a subject.

In some embodiments, constants a and b in Eq. 1 above may be determined by performing a calibration. The calibration may involve taking a reference blood pressure reading to obtain a reference blood pressure Pₒ, measuring the elapsed time Tₒ corresponding to the reference blood pressure, and then determining values for both of the constants a and b from the reference blood pressure and elapsed time measurement. Calibration may be performed at any suitable time (e.g., once initially after monitoring begins) or on any suitable schedule (e.g., a periodic or event-driven schedule).

In some embodiments, the calibration may include performing calculations mathematically equivalent to

$$\begin{matrix}a = {C}_{1} + \frac{{C}_{2} ( {P}_{o} - {C}_{1} )}{ln ( {T}_{o} ) + {C}_{2}} \\ and & ( 2 ) \\ b = \frac{{P}_{o} - {C}_{1}}{ln ( {T}_{o} ) + {C}_{2}} & ( 3 )\end{matrix}$$

to obtain values for the constants a and b, where C₁ and C₂ are parameters that may be determined, for example, based on empirical data.

In some embodiments, the calibration may include performing calculations mathematically equivalent to

a=Pₒ−(C₃Tₒ+C₄)·ln(Tₒ) (4)

and

b=C₃Tₒ+C₄, (5)

where a and b are first and second parameters and C₃ and C₄ are parameters that may be determined, for example, based on empirical data.

Parameters C₁, C₂, C₃, and C₄ may be predetermined constants empirically derived using experimental data from a number of different subjects. A single reference blood pressure reading from a subject, including reference blood pressure Pₒ and elapsed time Tₒ from one or more signals corresponding to that reference blood pressure, may be combined with such inter-subject data to calculate the blood pressure of a subject. The values of Pₒ and Tₒ may be referred to herein as a calibration point. According to this example, a single calibration point may be used with the predetermined constant parameters to determine values of constants a and b for the subject (e.g., using Eqs. 2 and 3 or 4 and 5). The subject's blood pressure may then be calculated using Eq. 1. Recalibration may be performed by collecting a new calibration point and recalculating the constants a and b used in Eq. 1. Calibration and recalibration may be performed using calibration device 180 of FIG. 1 or 280 of FIG. 2.

In some embodiments, multiple calibration points from a subject may be used to determine the relationship between the subject's blood pressure and one or more PPG signals. This relationship may be linear or non-linear and may be extrapolated and/or interpolated to define the relationship over the range of the collected recalibration data. For example, the multiple calibration points may be used to determine values for parameters C₁ and C₂ or C₃ and C₄ (described above). These determined values will be based on information about the subject (intra-subject data) instead of information that came from multiple subjects (inter-subject data). As another example, the multiple calibration points may be used to determine values for parameters a and b (described above). Instead of calculating values of parameters a and b using a single calibration point and predetermined constants, values for parameters a and b may be empirically derived from the values of the multiple calibration points. As yet another example, the multiple calibration points may be used directly to determine the relationship between blood pressure and PPG signals. Instead of using a predefined relationship (e.g., the relationship defined by Eq. 1), a relationship may be directly determined from the calibration points.

Additional examples of continuous and noninvasive blood pressure monitoring techniques are described in Chen et al., U.S. Pat. No. 6,599,251, which is hereby incorporated by reference herein in its entirety. The technique described by Chen et al. may use two sensors (e.g., ultrasound or photoelectric pulse wave sensors) positioned at any two locations on a subject's body where pulse signals are readily detected. For example, sensors may be positioned on an earlobe and a finger, an earlobe and a toe, or a finger and a toe of a subject's body.

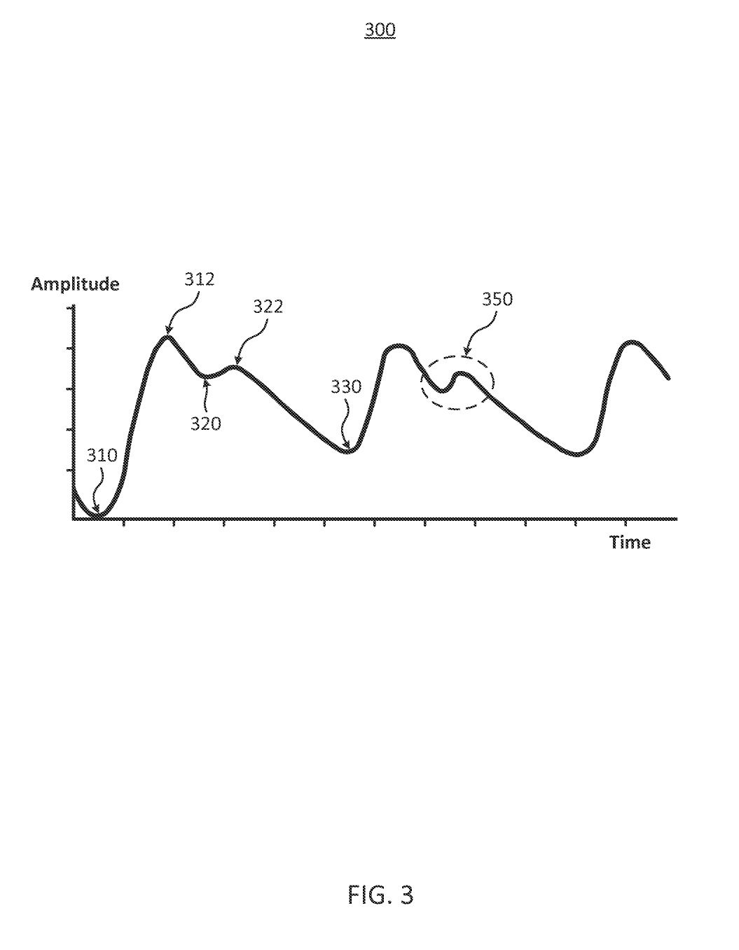

FIG. 3 shows an illustrative plot of a signal 300 that may be analyzed in accordance with some embodiments of the present disclosure. As discussed above, the processing equipment may receive a physiological signal from a subject. In some embodiments, the received physiological signal is an optical light signal, such as PPG signal 300. For purposes of brevity and clarity, and not by way of limitation, the present disclosure describes and depicts the received physiological signal as PPG signal 300. It will be understood that the received physiological signal and the depicted signal in FIG. 3 are not limited to a PPG signal and may correspond to a biopotential signal, pressure signal, impedance signal, temperature signal, acoustic signal, any other suitable physiological signal, or any combination thereof.

An illustrative PPG signal 300 is depicted in FIG. 3. The processing equipment may receive PPG signal 300 and may identify local minimum point 310, local maximum point 312, local minimum point 320, and local maximum point 322 in the PPG signal 300. The processing equipment may pair each local minimum point with an adjacent maximum point. For example, the processing equipment may pair points 310 and 312 to identify one segment, points 312 and 320 to identify a second segment, points 320 and 322 to identify a third segment and points 322 and 330 to identify a fourth segment. The slope of each segment may be measured to determine whether the segment corresponds to an upstroke portion of the pulse (e.g., a positive slope) or a downstroke portion of the pulse (e.g., a negative slope) portion of the pulse. A pulse may be defined as a combination of at least one upstroke and one downstroke. For example, the segment identified by points 310 and 312 and the segment identified by points 312 and 320 may define a pulse.

According to an embodiment, PPG signal 300 may include a dichrotic notch 350 or other notches (not shown) in different sections of the pulse (e.g., at the beginning (referred to as an ankle notch), in the middle (referred to as a dichrotic notch), or near the top (referred to as a shoulder notch). The processing equipment may identify notches and either utilize or ignore them when detecting the pulse locations. In some embodiments, the processing equipment may compute the second derivative of the PPG signal to find the local minima and maxima points and may use this information to determine a location of, for example, a dichrotic notch. Additionally, the processing equipment may interpolate between points in PPG signal 300 or between points in a processed signal using any interpolation technique (e.g., zero-order hold, linear interpolation, and/or higher-order interpolation techniques). Some pulse detection techniques that may be performed by the processing equipment are described in more detail in U.S. Patent Application Publication No. 2009/0326395, published Dec. 31, 2009, which is incorporated by reference herein in its entirety.

A physiological signal, for example, PPG signal 300, may be characterized by metrics indicative of the pulse wave morphology. These pulse wave morphology metrics and/or other metrics associated with characteristics of the physiological signal may indicate that a recalibration or a new measurement of a physiological parameter associated with the physiological signal should be performed and may be used to determine a change parameter. Metrics may include suitable signal values, signal morphologies, output values from suitable operations performed on the signal or other metrics, any other suitable mathematical characterizations, or any suitable combinations thereof. For example, metrics may include pulse wave area (PWA), geometric centroid of a pulse wave, rate of change computed at one or more points of a time series (e.g., derivative of any suitable order of a signal), statistics of a signal (e.g., mean, moment of any suitable order, regression parameters), offset of a signal from a baseline, interval of portion of a signal (e.g., length of upstroke), relative position of a fiducial point of a signal (e.g., dichrotic notch position), any other suitable metric or change thereof, or any suitable combinations thereof. For example, in some embodiments, the skewness (e.g., the standardized third central moment) of a pulse wave may be monitored.

Metrics may include mathematical manipulations of other metrics such as, for example, the value of an integral of a portion of a blood pressure measurement time series, the skewness of a derivative of a PPG signal, or any other suitable mathematical manipulations. In some embodiments, metrics may be computed from averaged, filtered, scaled, or otherwise processed physiological signals. For example, a derivative may be computed from a suitable ensemble average of pulse waves. The term “pulse wave” as used herein refers to a portion of a PPG signal corresponding to a physiological pulse.

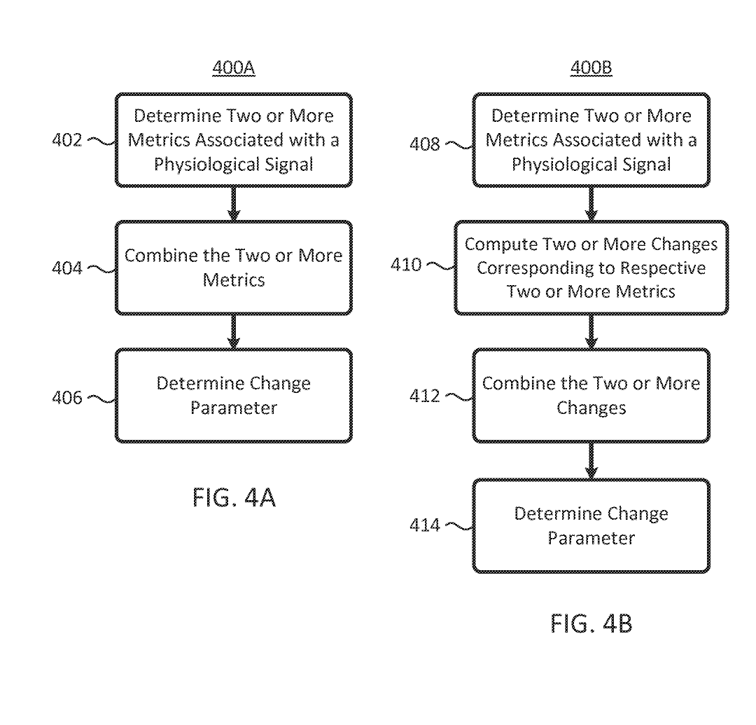

Processing equipment may determine a change parameter based at least in part on one or more metrics. In some embodiments, metrics may be associated with characteristics of the received physiological signal that indicate that a recalibration or a new measurement of a physiological parameter associated with the physiological signal should be performed. In an example, processing equipment may compute pulse wave morphology metrics using any of the foregoing techniques described above with reference to FIG. 3 and determine a change parameter based at least in part on the pulse wave morphology metrics. In some embodiments, the change parameter may be determined based on one metric. In some embodiments, the change parameter may be determined based on a combination of two or more metrics. FIGS. 4A and 4B, described below, illustrate different ways of determining a change parameter using two or more metrics.

FIGS. 4A and 4B show illustrative flow diagrams including steps for determining a change parameter in accordance with some embodiments of the present disclosure.

FIG. 4A shows illustrative flow diagram 400A including steps for determining a change parameter in accordance with some embodiments of the present disclosure.

At step 402, the processing equipment may determine two or more metrics associated with a physiological signal. The processing equipment may receive the physiological signal from a subject. The physiological signal may include one or more optical light signals, electrocardiograms, electroencephalograms, electrogastrograms, electromyograms, pulse rate signals, pathological signals, ultrasound signals, any other suitable biosignals, or any combination thereof. In some embodiments, the physiological signal corresponds to an optical light signal attenuated by a subject. In some embodiments, a light detector, such as detector 118 of FIG. 1, may receive the physiological signal. A light detector may detect light signals generated by light emitters that may have been partially attenuated by a subject before being detected. It will be understood that any suitable light detector or combination of light detectors may be used to detect the attenuated light signal. The amount of attenuation may correspond to, in the example of a pulse oximeter, a volume of blood or other tissue through which the light has travelled. In some embodiments, a monitor, such as monitor 114 of FIG. 1 or monitors 214 or 226 of FIG. 2, may receive the physiological signal. In some embodiments, the received physiological signal may have undergone signal processing before being received, such as any suitable band-pass filtering, adaptive filtering, closed-loop filtering, any other suitable filtering, or any combination thereof. In some embodiments, signal processing may be performed on the physiological signal after it has been received. It will be understood that the processing equipment may receive any suitable physiological signal, and it is not limited to receiving an optical light signal. For example, the received physiological signal may correspond to a biopotential signal, pressure signal, impedance signal, temperature signal, acoustic signal, any other suitable physiological signal, or any combination thereof.

In some embodiments, the processing equipment may determine two or more metrics associated with the received physiological signal. Metrics may include physiological metrics, for example, heart rate of a subject, morphology of the received physiological signal, metrics indicative of vascular resistance of a subject, variability metrics, metrics indicative of artifact in the received physiological signal, metrics indicative of changing systemic nervous activity, any other suitable metric associated with the received physiological signal or a determined physiological parameter associated with the physiological signal, and any combination thereof. In some embodiments, a metric may be associated with a characteristic of the received physiological signal that indicates that a recalibration or a new measurement of a physiological parameter associated with the physiological signal should be performed. In an example, the processing equipment may receive an optical light signal from a subject and determine a first metric, heart rate, and a second metric, pulse wave morphology indicative of vascular resistance. In some embodiments, pulse wave morphology metrics may be computed using any of the foregoing techniques described above with reference to FIG. 3.

At step 404, the processing equipment may combine the two or more metrics. The processing equipment may combine the metrics using, for example, a neural network, a polynomial equation, weighted summation, relative maximums, relative minimums, any other suitable technique, or any combination thereof. In some embodiments, a relative maximum or relative minimum may be determined from a comparison of the determined values of the two or more metrics.

At step 406, the processing equipment may determine a change parameter based at least in part on the combined two or more metrics. In some embodiments, the change parameter may correspond to the absolute change in the combined two or more metrics over time. In some embodiments, the change parameter may correspond to the relative change in the combined two or more metrics over time relative to a baseline value. For example, the processing equipment may determine a baseline value at a certain time and may compute the change parameter based on the amount by which the combined metrics differ from the baseline value over time. In some embodiments, the processing equipment may determine a change parameter based at least in part on the area under the curve formed by the combined two or more metrics over time. In some embodiments, the processing equipment may determine a change parameter based at least in part on a combination of the areas under the curves formed by each of the two or more metrics over time. In some embodiments, the change parameter is indicative of a likelihood of a change in a physiological parameter generated from the received physiological signal. In some embodiments, the change parameter is indicative of a magnitude of a change in a physiological parameter associated with the received physiological signal. In an example, the physiological parameter may be blood pressure, and the change parameter may be indicative of the likelihood of a change in the blood pressure of a subject since the last measurement of the subject's blood pressure (e.g., since the last direct measurement by blood pressure cuff inflation).

FIG. 4B shows illustrative flow diagram 400B including steps for determining a change parameter in accordance with some embodiments of the present disclosure.

At step 408, the processing equipment may determine two or more metrics associated with a physiological signal. The two or metrics may be determined as described in step 402 of FIG. 4A.

At step 410, the processing equipment may compute two or more changes corresponding to the respective two or more metrics. The processing equipment may compute a change in a corresponding metric using any suitable mathematical technique for calculating a change. In some embodiments, a change in a corresponding metric may be an absolute change over time or may be a relative change over time relative to a baseline value. In some embodiments, a change in a corresponding metric may be computed over any specified time period. In an example, the change may be computed over the time passed since the last measurement of a physiological parameter associated with the received physiological signal. In some embodiments, the physiological parameter may be blood pressure, and a change in a corresponding metric may be computed based on a current determined metric value and a metric value determined immediately following the last blood pressure measurement by cuff inflation.

At step 412, the processing equipment may combine the two or more changes corresponding to the respective two or more metrics. The processing equipment may combine the two or more changes using any suitable technique described with reference to combining the two or more metrics in step 404 of FIG. 4A.

At step 414, the processing equipment may determine a change parameter based at least in part on the physiological signal over time. In some embodiments, the processing equipment determines the change parameter based at least in part on the combination of the two or more changes corresponding to the two or more metrics. In some embodiments, the processing equipment may determine the change parameter based at least in part on the area under the curve formed by the combined two or more changes over time or on a combination of the areas under the curves formed by each of the two or more changes over time. In an example, the physiological parameter may be blood pressure, and the change parameter may be indicative of the magnitude of a change in the blood pressure of a subject since the last measurement of the subject's blood pressure (i.e., since the last direct measurement by blood pressure cuff inflation).

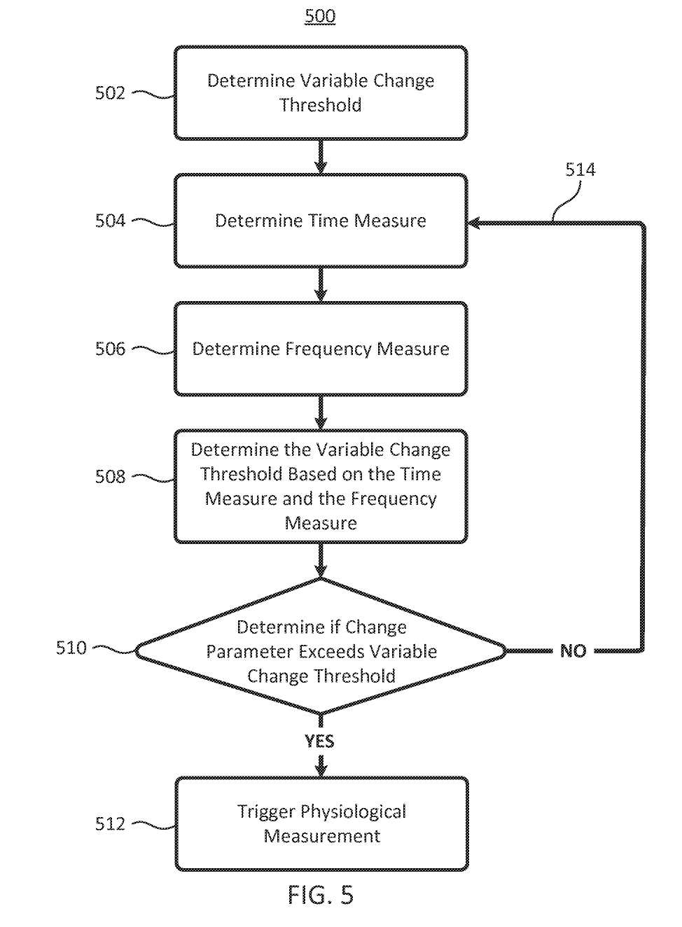

After the processing equipment determines the change parameter, it may compare the change parameter with a determined variable change threshold. The processing equipment may determine the variable change threshold based at least in part on a time measure and/or a frequency measure, as shown in FIG. 5.

FIG. 5 shows an illustrative flow diagram 500 including steps for determining a variable change threshold in accordance with some embodiments of the present disclosure.

At step 502, the processing equipment may determine a variable change threshold. In some embodiments, the processing equipment may determine the variable change threshold based at least in part on a predetermined fixed value. For example, a variable change threshold may be based on an acceptable number of blood pressure cuff inflations permitted over a period of time (e.g., 5 inflations). In some embodiments, the processing equipment may determine a variable change threshold based at least in part on a predetermined initial value. For example, the variable change threshold may be set to a high value immediately following a blood pressure cuff inflation (e.g., a high value consistent with a systolic blood pressure change of 30-40 mm Hg). In some embodiments, the processing equipment may determine a variable change threshold based at least in part on user input. User input may be entered using, for example, user inputs 130 of FIG. 1. User input may include, for example, subject-specific data, including gender, age, weight, height, medical history, predisposition to cardiac arrhythmia, medication information, any other suitable subject characteristic, or any combination thereof. For example, user input may include data indicating the subject is a 60-year-old female, and the processing equipment may determine the variable change threshold based at least in part on this user input. In some embodiments, the processing equipment may determine the variable change threshold by dynamically varying a threshold based on a proximity and/or frequency of past triggered measurements. In some embodiments, the processing equipment may determine a variable change threshold based at least in part on any specified number of measures, including a time measure, discussed with reference to step 504, and a frequency measure, discussed with reference to step 506.

At step 504, the processing equipment may determine a time measure. In some embodiments, the processing equipment may determine a time measure indicative of an amount of time that has passed since a measurement was triggered. For example, a time measure may be indicative of an amount of time that has passed since the last blood pressure cuff inflation. In some embodiments, the time measure may be indicative of a period of time between the triggering of a first measurement and a second measurement. In some embodiments, the time measure may be based on historical time data, including, for example, an average time interval between measurements, a maximum or minimum time interval between measurements, the amount of time that has passed since any specified previous measurement, any other suitable time data associated with one or more measurements, or any combination thereof.

At step 506, the processing equipment may determine a frequency measure. In some embodiments, the processing equipment may determine a frequency measure indicative of how frequently a measurement has been triggered. For example, the frequency measure may be indicative of how many times a blood pressure cuff inflation has been triggered over a specified period of time. In some embodiments, the frequency measure may be based on historical frequency data, including, for example, an average frequency of measurements, a maximum or minimum frequency of measurements, the frequency of measurements since any past point in time, any other suitable frequency data associated with one or more measurements, or any combination thereof.

At step 508, the processing equipment may determine the variable change threshold over time based at least in part on the time measure, the frequency measure, or both. In some embodiments, the processing equipment may determine a variable change threshold over time based on a time measure, which may be indicative of time passed since a physiological measurement or recalibration was triggered. A non-invasive blood pressure cuff inflation measurement may disrupt the readings of other physiological monitors connected to the subject. In some embodiments, a variable change threshold determined based on a time measure may be used to ensure that measurements or recalibrations are triggered for large changes over a short period of time and for lesser changes after a longer period of time so as to minimize interference with the readings of other instruments. In some embodiments, the processing equipment may determine a variable change threshold over time based on a frequency measure, which may be indicative of how frequently the measurement has been triggered. In some embodiments, a variable change threshold based on a frequency measure may be used to ensure that measurements or recalibrations are not triggered too frequently, so as to minimize subject discomfort and interference with other instrumentation. Accordingly, a variable change threshold determined based on both a time measure and a frequency measure may cause measurements or calibrations to be triggered at physiologically appropriate times while minimizing both discomfort for the subject and interference with the readings of other physiological monitors.

In some embodiments, as discussed above, the processing equipment may determine the variable change threshold over time based at least in part on the time measure. For example, the processing equipment may set a variable change threshold to a high initial value immediately following a previous blood pressure cuff inflation, and the processing equipment may gradually decrease the variable change threshold from the high initial value towards a zero value over the amount of time passed since the previous cuff inflation. In some embodiments, determining a variable change threshold based at least in part on a time measure may be accomplished by using any suitable technique for decreasing the value of a variable change threshold to zero over a finite period of time, for example, asymptotically decreasing the time threshold toward zero, decreasing using a step function, decreasing by a fixed or varying quantity after a certain amount of time has passed, decreasing using any function that converges to zero over a finite time period, any other suitable technique, or any combination thereof. In some embodiments, the variable change threshold may be decreased to a value greater than zero, but less than the initial value.

In some embodiments, the processing equipment may determine the variable change threshold over time based at least in part on the frequency measure. In an example, the processing equipment may determine a variable change threshold based on a frequency measure indicative of the number of blood pressure cuff inflations that have been triggered over a specified period of time. In another example, a frequency measure may be indicative of a maximum limit of 15 cuff inflations per hour for a particular subject and that 13 cuff inflations have been triggered over the last 45 minutes, and, based on this frequency measure, the processing equipment may increase the variable change threshold. As discussed below with respect to steps 510-512, a measurement may be triggered by a determination that a change parameter exceeds a variable change threshold. Thus, increasing the variable change threshold may reduce the frequency of triggered measurements, and in the above example, the frequency of cuff inflations may be reduced. Reducing the frequency of cuff inflations when the cuff inflations are being triggered too frequently may ensure that the subject's blood pressure may continue to be monitored over the full time period (e.g., 1 hour) without exceeding the maximum limit on the permissible frequency of cuff inflations.

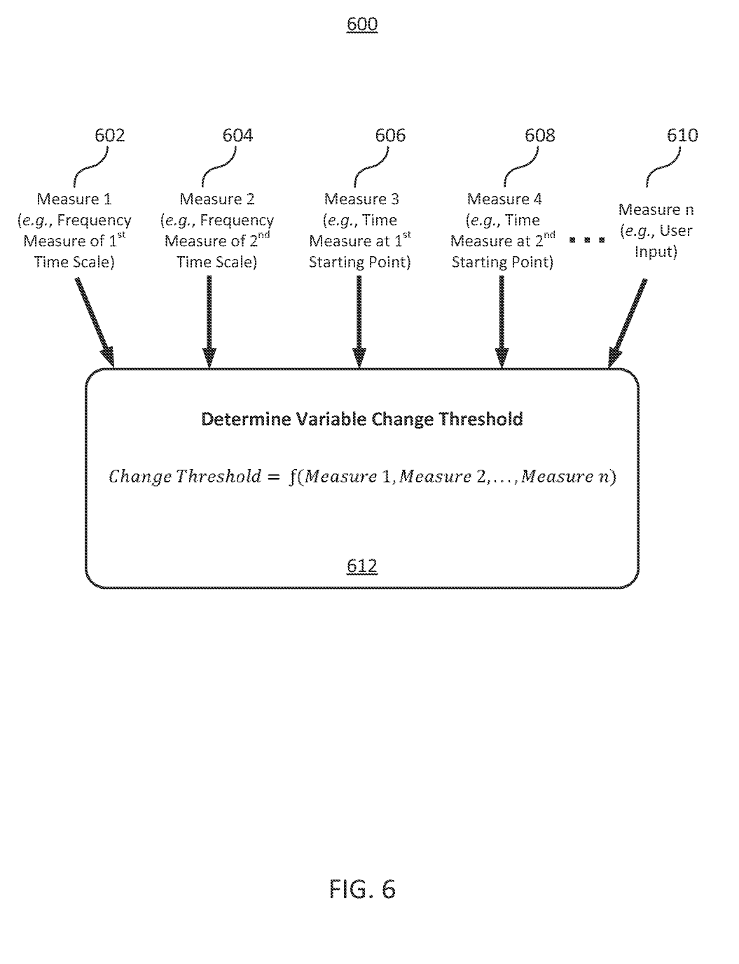

In some embodiments, the processing equipment may determine a variable change threshold over time based at least in part on the time measure and the frequency measure. In some embodiments, the processing equipment may set the initial change threshold based at least in part on the frequency measure and decrease the variable change threshold towards a lower value or a zero value over time based at least in part on the time measure. For example, the initial change threshold may be set to a higher value when the frequency measure is high and a lower value when the frequency measure is low. In some embodiments, the processing equipment may update the slope or amount of decay of the variable change threshold based on both the frequency measure and the time measure. For example, the processing equipment may determine the variable change threshold based on the time passed since a previous cuff inflation and based on the historical frequency of cuff inflations. Some embodiments of step 508 are further discussed with reference to FIG. 6.

For brevity and clarity, and not by way of limitation, some examples in the foregoing discussion of flow diagram 500 were explained with the measurement of a physiological parameter corresponding to direct measurement of blood pressure using a blood pressure cuff inflation system. It will be understood that the measurement of a physiological parameter is not limited to direct measurement by cuff inflation of blood pressure and may correspond to any suitable measurement of any suitable physiological parameter.

FIG. 6 is an illustrative block diagram 600 for determining a variable change threshold in accordance with some embodiments of the present disclosure. Processing block 612 may be implemented using any suitable processing equipment including, for example, microprocessor 148 shown in FIG. 1. Processing block 612 may receive any specified number of n measures. As depicted, processing block receives measures 602, 604, 606, 608, and 610. In some embodiments, measures 602, 604, 606, 608, and 610 may include any of the measures determined in steps 504 and 506 of FIG. 5. The processing equipment may determine the variable change threshold over time as a function of any specified number and combination of measures 602, 604, 606, 608, and 610. In some embodiments, the processing equipment may determine the variable change threshold over time based at least in part on one or more different time measures 606 and 608, one or more different frequency measures 602 and 604, or any combination thereof. In some embodiments, a frequency measure may be determined over different time scales. In the embodiment shown, measure 602 is a frequency measure of a first time scale and measure 604 is a frequency measure of a second time scale. For example, measure 602 may be a frequency of cuff inflations over the past thirty minutes, and measure 604 may be a frequency of cuff inflations over the past sixty minutes. In some embodiments, a time measure may be determined over different time windows or from different starting points in time. In the embodiment shown, measure 606 is a time measure at a first starting point and measure 608 is a time measure at a second starting point. For example, measure 606 may be time passed since the last measurement was triggered, and measure 608 may be time passed since the first of five measurements was triggered. In some embodiments, the processing equipment may determine the variable change threshold based on additional or alternative measures, including, for example, measure 610, which may be user input. User input may be entered using, for example, user inputs 130 of FIG. 1. User input may include, for example, subject-specific data, including gender, age, weight, height, medical history, predisposition to cardiac arrhythmia, medication information, any other suitable subject characteristic, or any combination thereof. For example, user input may include data indicating the subject is a 60-year-old female, and the processing equipment may determine the variable change threshold based at least in part on this user input. In some embodiments, measures 602, 604, 606, 608, and 610 may be selected based on user input. It will be understood that measures 602, 604, 606, 608, and 610 are presented for purposes of illustration and not by way of limitation. It will also be understood that measures 602, 604, 606, 608, and 610 may include any suitable measures determined using any suitable techniques, including, for example, determining measures based on short or long response times, differing time scales, differing time windows, differing starting points, any other suitable techniques for determining measures, and any combination thereof.

Referring back to FIG. 5, at step 510, the processing equipment may determine if the change parameter exceeds the variable change threshold. The processing equipment may use any suitable technique for comparing the value of the change parameter to the variable change threshold to determine if the change parameter exceeds the variable change threshold. In some embodiments, the change parameter may exceed the variable change threshold if it is determined to be equal to the variable change threshold.

At step 512, the processing equipment may trigger a physiological measurement when it is determined that the change parameter exceeds the variable change threshold. In some embodiments, the processing equipment may trigger a measurement of a physiological parameter associated with the received physiological signal. Physiological parameters may include, for example, one or more of blood pressure, cardiac output, preload, afterload, blood oxygen saturation (e.g., arterial, venous, or both), pulse rate, respiration rate, respiration effort, hemoglobin concentration (e.g., oxygenated, deoxygenated, and/or total), any other suitable hemodynamic parameters, any other suitable physiological parameters, or any combination thereof. For example, the change parameter may be determined from a PPG signal and the change parameter may correspond to changes in blood pressure. When the change parameter exceeds the variable change threshold, a non-invasive blood pressure reading may be triggered. In some embodiments, the processing equipment may trigger a calibration or recalibration of a physiological monitoring system based on the triggered measurement of a physiological parameter. For example, the processing equipment may trigger a recalibration of a continuous blood pressure calculation based on a triggered blood pressure measurement. The calibration or recalibration may be performed, for example, by calibration device 180 of FIG. 1 or calibration device 280 of FIG. 2. In some embodiments, the processing equipment may trigger an indicator injection for use in indicator dilution measurement of cardiac output. The cardiac output may be measured based at least in part on the triggered indicator injection. In some embodiments, the processing equipment may display a recommendation for a measurement, recalibration, or injection using, for example, display 120 of FIG. 1 or display 220 or display 228 of FIG. 2. The displayed recommendation may prompt the user to confirm or approve the recommendation. In response to a user input confirming or approving the recommendation (e.g., using user inputs 130 of FIG. 1), the processing equipment may trigger the measurement, recalibration, or injection.

When it is determined that the change parameter does not exceed the variable change threshold at step 510, the processing equipment may repeat the steps for determining the variable change threshold based at least in part on the time measure and/or the frequency measure. For example, the processing equipment may determine a new time measure at step 504, a new frequency measure at step 506, and determine the variable change threshold based at least in part on the new time measure and/or the new frequency measure at step 508. At step 510, the processing equipment may determine if the change parameter exceeds the determined change threshold, and based on the determination, either trigger the physiological measurement at step 512, or determine an updated variable change threshold starting at step 514.

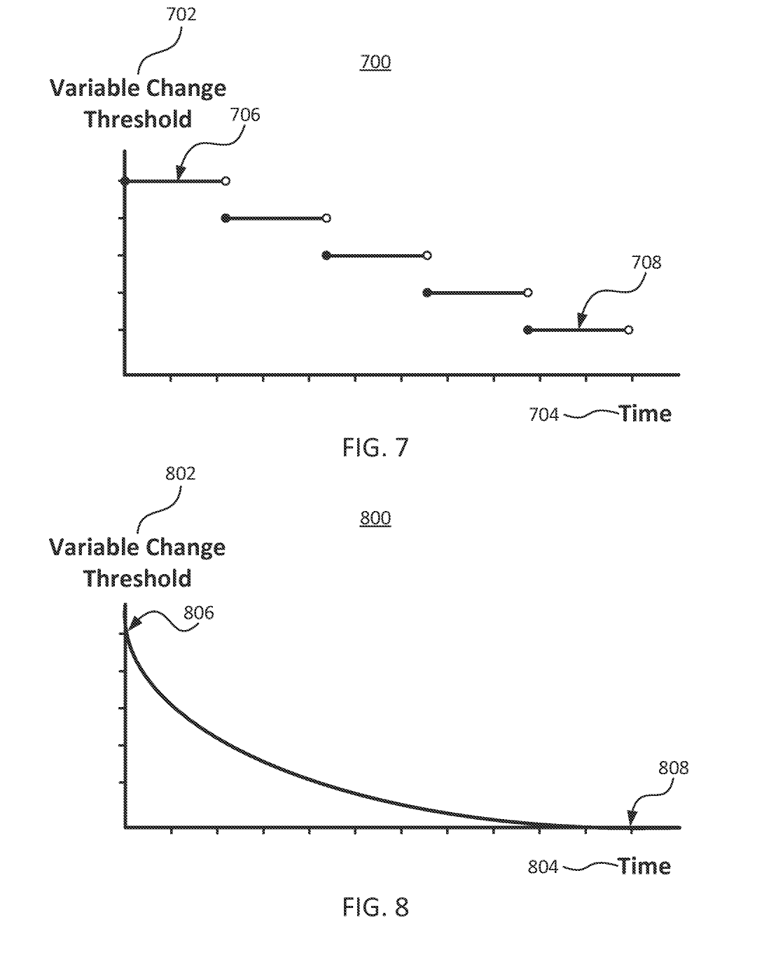

FIGS. 7 and 8 show illustrative plots of change thresholds in accordance with some embodiments of the present disclosure. It will be understood that the particular plots shown and the signals of those plots are merely exemplary.

FIG. 7 shows an illustrative plot 700 of a variable change threshold in accordance with some embodiments of the present disclosure. Vertical axis 702 of plot 700 corresponds to values of the variable change threshold and horizontal axis 704 corresponds to time. Plot 700 depicts the values of the variable change threshold decreasing from initial value 706 to value 708.

In some embodiments, a variable change threshold is determined over time as a function of time (i.e., time measure) and frequency (i.e., frequency measure). In the embodiment shown, plot 700 depicts the values of a variable change threshold decreasing as a step function over time from initial value 706 to value 708. For example, the variable change threshold value may decrease by a specified quantity every 2 minutes. As described above with respect to step 508 of FIG. 5, in some embodiments, initial value 706 may be determined based at least in part on a frequency measure, which may correspond to the frequency measure determined in step 506 of FIG. 5. The variable change threshold may be determined so that it decreases uniformly over time, as depicted in plot 700. In some embodiments, initial value 706 may occur at a point in time immediately following a triggered measurement of a physiological parameter. In some embodiments, the variable change threshold may be decreased towards a lower value or a zero value (i.e., value 708) until a measurement is triggered. When a change parameter is compared to the values of the variable change threshold depicted in plot 700, a larger change parameter (e.g., change parameter with value near or exceeding initial value 706) may exceed the variable change threshold after a short period of time, whereas a smaller change parameter (e.g., a change parameter with a value near or exceeding value 708) may exceed the variable change threshold after a longer period of time. Thus, a larger change parameter may more quickly trigger a measurement of a physiological parameter, and a smaller change parameter may trigger a measurement of a physiological parameter only after a longer amount of time has passed. In an example, the change parameter of a subject may increase to a very high value shortly after a measurement was triggered, which may correspond to a large deviation in the subject's blood pressure. The high-valued change parameter may be determined to exceed the variable change threshold within a very short period of time after the previous measurement, and a cuff inflation may be triggered to measure and update the subject's blood pressure.

FIG. 8 shows an illustrative plot 800 of a variable change threshold in accordance with some embodiments of the present disclosure. Vertical axis 802 of plot 800 corresponds to values of the variable change threshold and horizontal axis 804 corresponds to time. Plot 800 depicts the values of the variable change threshold decreasing from initial value 806 to value 808.

In some embodiments, a variable change threshold is determined over time as a function of time and frequency. In the embodiment shown, plot 800 depicts the values of the variable change threshold decreasing asymptotically over time from initial value 806 to near-zero value 808. For example, as described above with respect to step 508 of FIG. 5, in some embodiments, initial value 806 may be determined based at least in part on a frequency measure, which may correspond to the frequency measure determined in step 506 of FIG. 5. The variable change threshold may be determined so that it decreases non-uniformly over time, as depicted in plot 800. In some embodiments, initial value 806 may occur at a point in time immediately following a triggered measurement of a physiological parameter. In some embodiments, change threshold may be decreased towards a zero value until a measurement is triggered, which may correspond to the time at value 808. In an example, a subject may possess a low tolerance for blood pressure cuff inflations, so a maximum number of inflations may be imposed for a period of time. The subject may exhibit a small deviation in measured blood pressure, which may be reflected in a corresponding low-valued change parameter. The low-valued change parameter may be determined to exceed the variable change threshold only after a period of time has passed, and the cuff inflation may be triggered only as necessary, rather than repeatedly after a short, fixed time interval, which may result in excessive discomfort for the subject. As compared to the variable change threshold in FIG. 7, the variable change threshold shown in FIG. 8 may be more sensitive to variations in the change parameter. For example, in plot 700, a change parameter may not exceed a variable change threshold set to value 706 until the variable change threshold decreases to the next step after a period of time has passed. Whereas, in in plot 800, the same change parameter may exceed the variable change threshold after a shorter period of time has passed, as the variable change threshold decreases more quickly from value 806 than the variable change threshold parameter does in plot 700.

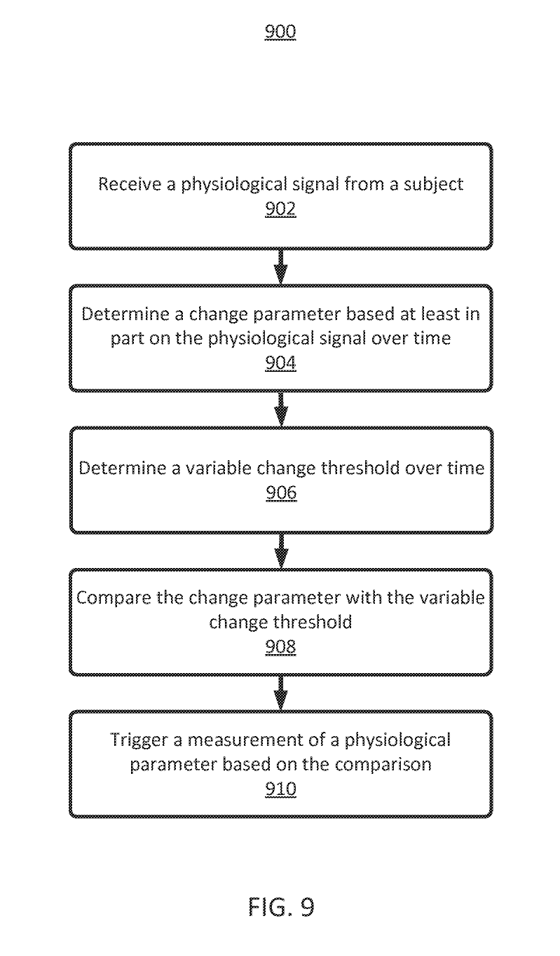

FIG. 9 shows an illustrative flow diagram 900 including steps for triggering a physiological measurement in accordance with some embodiments of the present disclosure.

At step 902, the processing equipment may receive a physiological signal from a subject. Physiological signals may include optical light signals, electrocardiograms, electroencephalograms, electrogastrograms, electromyograms, pulse rate signals, pathological signals, ultrasound signals, any other suitable biosignals, or any combination thereof. In some embodiments, the physiological signal corresponds to an optical light signal attenuated by a subject. In some embodiments, a light detector, such as detector 118 of FIG. 1, may receive the physiological signal. A light detector may detect light signals generated by light emitters that may have been partially attenuated by a subject before being detected. It will be understood that any suitable light detector or combination of light detectors may be used to detect the attenuated light signal. The amount of attenuation may correspond to, in the example of a pulse oximeter, a volume of blood or other tissue through which the light has travelled. In some embodiments, a monitor, such as monitor 114 of FIG. 1 or monitors 214 or 226 of FIG. 2, may receive the physiological signal. In some embodiments, the received physiological signal may have undergone signal processing before being received, such as any suitable band-pass filtering, adaptive filtering, closed-loop filtering, any other suitable filtering, or any combination thereof. In some embodiments, signal processing may be performed on the physiological signal after it has been received. It will be understood that the processing equipment may receive any suitable physiological signal, and it is not limited to receiving an optical light signal. For example, the received physiological signal may correspond to a biopotential signal, pressure signal, impedance signal, temperature signal, acoustic signal, any other suitable physiological signal, or any combination thereof.

At step 904, the processing equipment may determine a change parameter based at least in part on the physiological signal over time. In some embodiments, the processing equipment may determine a change parameter based at least in part on one metric (e.g., a continuous blood pressure calculation). In some embodiments, as described above with respect to step 406 of FIG. 4A and step 414 of FIG. 4B, the processing equipment may determine a change parameter based at least in part on a combination of two or more metrics and/or a combination of two or more changes corresponding to two or more metrics. In some embodiments, more than one change parameter may be determined for a particular physiological signal using any of the foregoing techniques. In some embodiments, the processing equipment may determine a change parameter, based on the physiological signal, by assessing a measure of change over time.

At step 906, the processing equipment may determine a variable change threshold. The variable change threshold may be determined as described in step 502 or step 508 of FIG. 5. In some embodiments, the processing equipment may determine the variable change threshold over time. As described above, with reference to processing block 612 of FIG. 6, the variable change threshold may be determined over time based at least in part on any specified number of measures, including one or more frequency measures, one or more time measures, user input, any other suitable measures, or any combination thereof. In some embodiments, the variable change threshold may be determined dynamically. In some embodiments, the processing equipment may determine the variable change threshold based at least in part on dynamically determined measures and/or on historical data. In some embodiments, more than one variable change threshold may be determined for a particular physiological signal using any of the foregoing techniques. In some embodiments, the more than one variable change thresholds may correspond to sensitivity levels. In some embodiments, the processing equipment may determine which of the more than one variable change thresholds to compare with the change parameter based on a sensitivity level, which may be set based on user input.

At step 908, the processing equipment may compare the change parameter with the variable change threshold. The processing equipment may determine if the change parameter exceeds the variable change threshold. The processing equipment may use any suitable technique for comparing the value of the change parameter to the variable change threshold to determine if the change parameter exceeds the variable change threshold. In some embodiments, the change parameter may exceed the variable change threshold if it is determined to be equal to the variable change threshold.

At step 910, the processing equipment may trigger a measurement of a physiological parameter when the change parameter exceeds the variable change threshold. In some embodiments, the processing equipment may trigger a measurement of a physiological parameter associated with the received physiological signal, as described with reference to step 512 of FIG. 5. Physiological parameters may include, for example, one or more of blood pressure, cardiac output, preload, afterload, blood oxygen saturation (e.g., arterial, venous, or both), pulse rate, respiration rate, respiration effort, hemoglobin concentration (e.g., oxygenated, deoxygenated, and/or total), any other suitable hemodynamic parameters, any other suitable physiological parameters, or any combination thereof. In some embodiments, the processing equipment may not trigger a physiological measurement until after a minimum amount of time has passed since the last physiological measurement. For example, the processing equipment may not trigger a blood pressure cuff inflation measurement before a minimum of two minutes has passed since the last cuff inflation, even if it is determined that the change parameter exceeds the variable change threshold. As another example, steps 902-910 may not be performed until the minimum amount of time has passed. In some embodiments, the processing equipment may automatically trigger a physiological measurement after a maximum amount of time has passed since the last physiological measurement. For example, the processing equipment may trigger a blood pressure cuff inflation measurement after a maximum of twenty minutes has passed since the last cuff inflation, even if it is determined that the change parameter does not exceed the variable change threshold. In some embodiments, the maximum and minimum amounts of time may be set based on predetermined values, user input, any other suitable data, or any combination thereof.

In some embodiments, where more than one change parameter and more than one change threshold have been computed, as described above with reference to steps 904 and 906, the processing equipment may compare each change parameter to a respective change threshold. In some embodiments, if one or more of the change parameters exceed the respective one or more change thresholds, then a measurement may be triggered, as described in step 910. It will be understood that any other suitable method may be used for comparing two or more change parameters to two or more respective change thresholds and for determining whether to trigger a measurement. For example, the two or more change parameters may be combined into a final change parameter, and the two or more change thresholds may be combined into a final change threshold, and the final change parameter may be compared to the final change threshold to determine whether a measurement to trigger a measurement.

It will be understood that the steps above are exemplary and that in some implementations, steps may be added, removed, omitted, repeated, reordered, modified in any other suitable way, or any combination thereof.

The foregoing is merely illustrative of the principles of this disclosure, and various modifications may be made by those skilled in the art without departing from the scope of this disclosure. The above-described embodiments are presented for purposes of illustration and not of limitation. The present disclosure also can take many forms other than those explicitly described herein. Accordingly, it is emphasized that this disclosure is not limited to the explicitly disclosed methods, systems, and apparatuses, but is intended to include variations to and modifications thereof, which are within the spirit of the following claims.

What is claimed:

1. A system for triggering a blood pressure measurement of a subject comprising:
an input configured for receiving a physiological signal representative of light passing through a subject's tissue; and
one or more processors configured for:
determining a change parameter based at least in part on a measure of change of the physiological signal over time;
comparing the change parameter to a threshold;
triggering a new blood pressure measurement when the change parameter passes the threshold; and
dynamically varying the threshold based on a proximity or frequency of past triggered blood pressure measurements.

2. The system of claim 1, wherein the one or more processors are further configured for:
determining two or more metrics associated with the physiological signal; and
combining the two or more metrics to determine the change parameter.

3. The system of claim 2, wherein combining the two or more metrics comprises:
computing two or more changes corresponding to the respective two or more metrics; and
combining the two or more changes to determine the change parameter.

4. The system of claim 1, wherein the change parameter of the physiological signal is based at least in part on a pulse wave morphology metric.

5. The system of claim 4, wherein the pulse wave morphology metric is selected from the group consisting of pulse wave area, geometric centroid, rate of change, a statistical signal metric, signal offset, an interval metric, fiducial point position, skewness, and combinations thereof.

6. The system of claim 1, wherein the change parameter is based at least in part on a physiological metric of the physiological signal.

7. The system of claim 6, wherein the physiological metric is selected from the group consisting of heart rate, blood pressure, oxygen saturation, vascular resistance, a variability metric, an artifact metric, a nervous activity metric, and combinations thereof.

8. The system of claim 1, wherein the one or more processors are further configured for determining a physiological parameter based on the physiological signal, and wherein the change parameter is indicative of a likelihood of a change in the physiological parameter.

9. The system of claim 8, wherein the physiological parameter comprises continuous blood pressure.

10. The system of claim 1, wherein the one or more processors are further configured for recalibrating a continuous blood pressure calculation based at least in part on the triggered blood pressure measurement.

11. The system of claim 1, wherein dynamically varying the threshold comprises varying the threshold according to a step function.

12. The system of claim 1, wherein dynamically varying the threshold comprises continuously decreasing the threshold over time.

13. A system for triggering a physiological measurement of a subject comprising:
an input configured for receiving a physiological signal from the subject; and
one or more processors configured for:
determining a change parameter based at least in part on the physiological signal over time;
determining a variable change threshold over time;
comparing the change parameter with the variable change threshold; and
triggering a measurement of a physiological parameter based on the comparison.

14. The system of claim 13, wherein determining a change parameter comprises:
determining two or more metrics associated with the physiological signal; and
combining the two or more metrics to determine the change parameter.

15. The system of claim 14, wherein combining the two or more metrics comprises:
computing two or more changes corresponding to the respective two or more metrics; and
combining the two or more changes.

16. The system of claim 13, wherein the physiological parameter is one or more of blood pressure, cardiac output, preload, and afterload.

17. The system of claim 13, wherein the physiological parameter is blood pressure, the one or more processors are further configured for recalibrating a continuous blood pressure calculation based on the triggered blood pressure measurement.

18. The system of claim 13, wherein determining the variable change threshold over time comprises:
determining a time measure indicative of an amount of time that has passed since the measurement was triggered; and
determining the variable change threshold based at least in part on the time measure.

19. The system of claim 13, wherein determining the variable change threshold over time comprises:
determining a frequency measure indicative of how frequently the measurement has been triggered; and
determining the variable change threshold is based at least in part on the frequency measure.

20. The system of claim 13, wherein determining the variable change threshold over time comprises:
determining a time measure indicative of an amount of time that has passed since a measurement was triggered;
determining a frequency measure indicative of how frequently the measurement has been triggered; and
determining the variable change threshold based at least in part on the frequency measure and the time measure.

21. The system of claim 13, wherein determining the variable change threshold over time comprises determining the variable change threshold over time based at least in part on user input.

22. The system of claim 13, wherein the physiological parameter is cardiac output, and wherein triggering a measurement comprises:
triggering an indicator injection based at least in part on the comparison; and
measuring a cardiac output of the subject based at least in part on the triggered indicator injection.

23. A system for triggering a physiological measurement of a subject, comprising:
processing equipment configured for:
receiving a photoplethysmography signal from a subject;
based on the photoplethysmography signal, assessing a measure of change over time;
comparing the measure of change to a dynamic threshold; and
triggering a new physiological measurement based on the comparison.
